(19) United States
(12) Patent Application Publication
(10) Pub. No.: US 2006/0217394 A1
(43) Pub. Date: Sep. 28, 2006

(54) Treatment of anhedonia

(75) Inventors: Donald L. Nathanson, Wynnewood, PA (US); Marc K. Samet, Broomall, PA (US)

Correspondence Address: MORGAN, LEWIS & BOCKIUS LLP, 1701 MARKET STREET, PHILADELPHIA, PA, 19103-2921, US

(73) Assignee: Silvan S. Tomkins Institute, Inc.

(21) Appl. No.: 11/360,969

(22) Filed: Feb. 23, 2006

Related U.S. Application Data
(60) Provisional application No. 60/655,343, filed on Feb. 23, 2005.

(51) Int. Cl. A61K 31/496; A61K 31/445; A61K 31/405; A61K 31/165; A61K 31/137

(52) U.S. Cl. 514/253.04; 514/419; 514/317; 514/649; 514/618

(57) ABSTRACT

A therapeutic method for the enhancement of mood in patients afflicted with iatrogenic anhedonia secondary to chronic increases in the availability and/or efficiency of serotonin, with or without increases in the availability and/or efficiency of norepinephrine.

### STATEMENT OF RELATED CASES

This application claims priority to U.S. Provisional Application No. 60/655,343 filed on Feb. 23, 2005 which is hereby incorporated by reference in its entirety.

### FIELD OF THE INVENTION

The present invention generally relates to a therapeutic method comprising administering a pharmaceutical agent capable of directly and/or indirectly increasing the availability and/or efficiency of dopaminergic activity to a subject (e.g., a patient) afflicted with anhedonia.

### BACKGROUND OF THE INVENTION

The discovery that chlorpromazine (originally developed as an antihistamine) was useful in major psychiatric disorders, and the subsequent investigation of its pharmacological activity, led to an understanding of the importance of neurotransmitter imbalance in mental diseases. Contemporary clinical wisdom now takes for granted that alterations of neurotransmitters, for example, catecholaminergic and serotonergic neurotransmitter systems, are responsible for the circuitry distortions associated with affective disorders. Among the hosts of agents found useful in the treatment of mental disorders are the antidepressant drugs, a class of medications that ameliorate the symptoms of depression. Among the first widely available agents were such tricyclic antidepressants as imipramine, nortryptyline, doxepin, amitriptyline, and desipramine. Investigation of the tricyclic antidepressants defied reuptake inhibition of norepinephrine and serotonin as primary mechanisms of antidepressant action. The observation that tricyclic antidepressants modulate both adrenergic and serotonergic transmission stimulated the search for compounds that would selectively inhibit the reuptake of either neurotransmitter alone.

Zimelidine, the first selective serotonin reuptake inhibitor (“SSRI”), was developed as a result of the finding that certain antihistamines did inhibit the uptake of serotonin. Later, the SSRI fluoxetine hydrochloride (PROZAC®) was identified as a more potent and better tolerated treatment for depression. Other SSRIs include paroxetine hydrochloride (PAXIL®) and sertraline hydrochloride (ZOLOFT®). Early institution of SSRI treatment has been found to produce dramatic increases in the frequency of successful treatment outcome. Research later showed that medications which inhibit the reuptake of both serotonin and norepinephrine (SNRIs), such as venlafaxine and duloxetine, were also very effective antidepressant agents.

Alterations in the levels of other central nervous system neurotransmitters have also been shown to produce profound effects on emotional state. For example, early experiments using reserpine to deplete central nervous system stores of catecholamines demonstrated the significance of dopamine in central nervous system function.

Standard psychiatric practice takes for granted that complete and lasting remission of chronic depressive illness usually requires from six months or more of steady treatment. Although reasonable, this common belief ignores the reality that a great many patients will remain on chronic dosing regimens for several years after the initiation of treatment. Within the Diagnostic and Statistical Manual of Mental Disorders, this cohort would be coded as 296.35 (chronic). Indeed, it is likely that those who recover from a first episode of depression quickly and permanently represent a genome quite different from those who require intermittent, long term, or lifetime treatment for their depression.

Counter to the expectations of those in the field, recent research has associated the use of acute SSRI dosing regimens in depressed patients not just with decreases in the serotonin transporter (SERT) but also with an increase in detectable dopamine transporters (“DAT”). SERT, the protein responsible for the reuptake of serotonin, acts therefore to terminate the activity of serotonin. Both synthesis and expression of DAT, which is of similarly critical significance in the clearance of dopamine, are influenced by dopamine (“DA”) receptor activity. It is noteworthy that these reciprocal changes in SERT and DAT, and thus alterations in the availability and efficiency of both serotonergic and dopaminergic transmission, have been demonstrated after only 8 weeks of SSRI treatment in both Major Depressive Disorder and Obsessive Compulsive disorder.

### SUMMARY OF THE INVENTION

It has been discovered that anhedonia, and in certain embodiments anhedonia associated with long term treatment with one or more SSRIs and/or SNRIs and which treatment produces an increase in serotonin availability and/or transmission efficiency with or without similar increases in the efficiency and availability of norepinephrine (referred to herein as “iatrogenic anhedonia”), may be treated with agents capable of directly or indirectly increasing the availability of dopamine and/or the efficiency of its activity.

More broadly, it has been found that persons suffering from or prone to anhedonia and anhedonia associated with chronic upregulation of serotonin, whether produced by genetic predisposition, SSRI drugs, SNRI drugs, plant derivatives, or by medications that cause quantitatively similar increases in serotonin availability and/or transmission efficiency with or without similar increases in the efficiency and availability of norepinephrine, can be treated for the specific anhedonia defined herein by the administration of agents capable of directly and/or indirectly increasing the availability of dopamine and/or directly or indirectly increasing the efficiency of dopaminergic activity.

While not limited to such examples, agents useful for effectuating an increase of dopaminergic activity and/or efficiency in the central nervous system include but may not be limited to bupropion, amphetamine salts, methylphenidate, nefazodone, quetiapine, ropinerole, and aripiprazole alone or in variable combinations, as well as derivatives thereof, as described in more detail below.

In one embodiment a treatment method is identified that includes the administration to iatrogenic anhedonic patients of one or more pharmaceutical substances that facilitate dopaminergic transmission. In this embodiment, the treatment regimens include agents that directly and/or indirectly bind to or activate dopaminergic receptors and/or dopamine transporters, optimize dopaminergic transmission efficiency, and/or inhibit the removal of dopamine.

In another embodiment, agents that either directly and/or indirectly enhance dopamine receptor activation and/or directly or indirectly increase the release and/or availability of dopamine are pharmaceutically active agents that when administered to patients in appropriate pharmaceutical doses of formulations that will effectively treat the anhedonia defined herein.

In yet another embodiment, methods are disclosed for the discovery of agents that enhance the ability of dopamine to stimulate activity through either direct and/or indirect mechanisms of action. The prediction of agents enhancing dopaminergic transmission efficiency therefore facilitates a method for the effective identification and development of agents and modalities useful in the amelioration of any such anhedonia caused by the prolonged upregulation of serotonin transmission efficiency.

In one embodiment, a method of treatment of the invention includes the steps of identifying the condition of iatrogenic anhedonia in a patient who had been treated with a first pharmaceutical agent that increases serotonin, and then therapeutically increasing dopaminergic activity in the patient by administering a second pharmaceutical agent or agents in amounts effective to ameliorate the iatrogenic anhedonia.

In another embodiment, a method for the treatment of iatrogenic anhedonia in a patient currently being treated for depression with a first pharmaceutical agent known to increase the availability or efficiency of serotonin, includes the steps of continuing the treatment of the patient with the first pharmaceutical agent known to increase the availability or efficiency of serotonin, and increasing dopaminergic activity in the patient by administering one or more pharmaceutical agents in an amount effective to ameliorate the iatrogenic anhedonia.

In yet another embodiment, a method for the treatment of iatrogenic anhedonia that includes the steps of identifying a patient as having iatrogenic anhedonia and then treating the patient with a pharmaceutical agent or agents that enhance dopaminergic availability and/or efficiency, and modulating doses of the pharmaceutical agent or agents that enhance dopaminergic availability and/or efficiency based upon clinical presentation and therapeutic responses seen in the patient.

Other features and advantages of the invention will be apparent from the following detailed description.

### DETAILED DESCRIPTION

The latest edition of the Diagnostic and Statistical Manual of Mental Disorders (DSM-IV-TR) published by the American Psychiatric Association and generally accepted as the main diagnostic reference for those practiced in the art of psychopharmacology does not recognize that pharmacologic treatment itself can produce a chronic and debilitating condition. The recognition that anhedonia may be caused by major depressive disorder treatment itself, hence “iatrogenic anhedonia,” rather than the underlying disease state has led to a development of a diagnosis and treatment regimen for the condition described herein.

The new disease target described herein involves forms of anhedonia defined and described in paragraphs below. Also presented herein is a treatment of anhedonia involving or caused by one or more agents that increase serotonergic transmission efficiency.

In the present invention, a therapeutic composition and method are provided for the enhancement of mood in patients afflicted with anhedonia. In one embodiment, as defined herein, iatrogenic anhedonia results from the chronic administration of any agent that produces elevation in serotonin availability and/or efficiency. The therapeutic regimen comprises a combination of agents capable of directly and/or indirectly increasing dopaminergic transmission efficiency.

In one embodiment, the present invention is directed to the diagnosis and treatment of patients suffering with a major depressive disorder and who had been managed successfully by therapies that elevate serotonin or increase serotonergic efficiency (with or without increases in the efficiency of norepinephrine).

As used herein, the terms “agent” or “agents” refer to pharmaceutically active compounds.

As used herein, the term “about” refers to + or −10% of the value referenced inclusive of the value referenced.

### I. Diagnosis of Iatrogenic Anhedonia and Spontaneous Anhedonia

In one embodiment, patients receiving one or more agents that increase the availability and/or efficiency of serotonin (with or without increases in the availability or efficiency of norepinephrine) are subsequently evaluated for the presence of the anhedonic symptoms defined herein, as described below.

In certain embodiments, such agents which induce iatrogenic anhedonia after chronic or long term administration (i.e., six months or more) include antidepressants. In another embodiment, such agents include SSRIs and/or SNRIs. In another embodiment, such agents include one or more of: diazobicyclooctane; tropane alkaloids which have the 8-azabicyclo[3.2.1]octane nucleus, AC-90179 [2-(4-Methoxyphenyl)-N-(4-methyl-benzyl)-N-(1-methyl-piperidin-4-yl)-acetamide Hydrochloride]: Spiro[9,10-dihydroanthracene]-9,3′-pyrrolidine; pyrazole; valdoxan; radafaxine; GW372475; NS2359; VPI-013, OPC-14523; elzasonan, CP-448187; Vilazodone 5-{4-[4-(5-cyano-3-indolyl)-butyl]-1-piperazinyl}-benzofuran-2-carboxamide; EMD 68843; Lu AA21004; SEP-225289; SLV 310,313; SSR 181507; GW773812 RTI 120; GBR 12909; imipramine; amitryptaline; trimipramine; doxepin; desipramine; nortriptyline; protriptyline; amoxapine; moprotline; reboxitine; fluoxetine; nefazodone; Zoloft; paroxetine; citalopram; fluvoxemine; duloxetine; escitalopram; venlafazine; desvenlafaxine; nisoxetine; KKHA-761 1-[4-[3-(3,4-dimethoxy-phenyl)-isoxazol-5-yl]-butyl-4-(2-methoxy-phenyl)-piperazine; 2-(4-methoxy-phenyl)-N-(4-methyl-benzyl)-N-(1-methyl-peiperidine-4yl)-acetamide; SB258585 [4-iodo-N-{4-methoxy-3-(4 methyl-piperazine-1-yl)-phenyl]-benzene sulfonamide; [6-fluror-10-[3-(2methoxyethyl)-4-methyl-piperazine-1-yl]-2-methyl-4H-3-thia-4,9-diaza-benzo[f]azulene]; M100907 R-(+)-alpha-(2,3,dimethoxyphenyl)-1-1[2-(4-flurorphenylethyl]-4-piperidine; 1-2-5-dimethoxy-4-bromophenol-2-aminopropane; SSR 181507; SB-243213; NRA0562; cyamameazine; bionanaserin (AD5423); 1-heteroaryl-4-[omega-(1H-indole-3-yl)alkyl]piperazines; SR463349; SSR504734; RS 102221; 8-[5-(2,4-dimethoxy-5-(4-triflouromethylphenylsulfon-amido)phenyl-5-oxopentyl]-1,3,8-triazaspiro[4.5]decane-2-4-dione; DOV 21,947; [(+)-1-(3,4dichlorophenyl)₃-azabiocyclo-[3.1.0]hexane; (S)-(−)4-[3-fluorophenoxy)-phenyl]-methyl-piperidine F-98214-TA; cis-fused 2-N,N-dimethylaminomethyl-2,3,3a,12b-tetrahydrodibenzo[b,f]furo[2,3-d]oxepin; DU-127090; 5-(3-[((2S)-1,4-benzodioxan-2-ylmethyl)amino]propoxy)-1,3-benzodioxole; N-4-aryl-piperazinyl-N′-ethyl-5,6,7,8-tetrahydropyrido[4′, 3′:4,5]thieno[2,3-d]pyrimidin-4(3H)-one; 2-[4-(2-methoxyphenyl)piperazin-1-yl]-N-(6-nitro-2-quinolyl)ethylamine; 1-(5-bromo-1,2,3,4-tetrahydronaphthalen-1-yl)-3-[4-(2-methoxyphenyl)-piperazin-1-yl]-1-propanone 2-[4-(2-methoxyphenyl)piperazin-1-yl]-N-(6-nitro-2-quinolyl)ethylamine 1-(5 bromo-1,2,3,4-tetrahydronapthalen-1-yl)-3-[4-(2-methoxyphenyl)-piperazine-1-yl]-1-propanone; indole cyclopropylmethylamine; 5-Chloroindole 699929; benzodioxanylpiperazine derivative lecozotan; (2R,4R)-4-hydroxy-2-[2-[2-[2-(3-methoxy)phenyl]ethyl]phenoxy]ethyl-1-methylpyrrolidine (R-96544); (2R,4R)-4-lauroyloxy-2-[2-[2-[2-(3-methoxy)phenyl]ethyl]phenoxy]ethyl-1-methylpyrrolidine (R-10244) N-4-aryl-piperazinly-N′-ethyl-5,6,7,8-tetrahydropyroido[4′,3′:4,5]thieno[2,3-d]pyrimidin-4(3H0)-one; 1-[4-amino-5-chloro-2-(3,5-dimethoxyphenil)methyloxy]-3-[1-[2-methylsulfonylamino]piperidine-4-yl]propan-1-one (RS 39604) RS42358-197; N-(1-ethyl-4-methylhexahydro-1,4-diazepin-6-yl)pyridine-3-carboxaides; (1S,4S)-4-(3,4,-dichlorophenyl)N-methyl-1,2,3,4-tetrahydro-1-naphthaleneamine; bicyclic heterarylpiperazine; N-[(3S)-1-benzylpyrrolidin-3-yl]-(2-thenyl)benazmides; PRX-00023; PRX03140; PRX-07037; risperidone; olanzapine; mirtazapine; mianserin; flesinoxan; buspirone; gepirone or gepirone er; ipsapirone; tandospirone; trazodone; triperidol; bionanserin; pindolol; ketanserin; 1-phenyl-1,2,3,4-tetrahydroisoquinolines and related 5,6,8,9-tetrahydro-13bH-dibenzo[a,h]quinolizines; (+/−)-7-chloro-8-hydroxy-3-[6-(N,N-dimethylamino)-hexyl]-1-phenyl-2,3,4,5-tetrahydro-1H-3-benzazepine; (+)-7-chloro-8-hydroxy-3-[11C]methyl-1-phenyl-2,3,4,5-tetrahydro-1H-3-benzazepine cis-10-hydroxy-4-n-propyl-1,2,3,4,4a,5,6,10b-octahydrobenzo[f]quinoline; 7-chloro-8-hydroxy-1-phenyl-2,3,4,5-tetrahydro-1H-3-methylbenzazepine (SCH 23390);([+]-2(3-chloro-6-methyl-8-phenyl-5,6,7,8-tetrahydro-4H-thieno[2,3d]azepin-2-yl)propan-2-ol); ([S]-6-chloro-1-[2,5-dimethoxy-4-propylbenzyl]-7-hydroxy-2-methyl-1,2,3,4-tetrahydroisoquinoline; ([R]-7-chloro-8-hydroxy-2,3,4,5-tetrahydro-3-methyl-1-phenyl-1H-3-benzazepine; cis- and trans-4-n-propyl-1,2,3,4,4a,5,6,10b-cetahydrobenzo[f]quinolines; (1S,2R)-8-Hydroxy-1-methyl-2-(dipropylamino)tetralins; cis-(1S,2R)-8-hydroxy-1-methyl-2-(di-n-propylamino)tetralin; monophenolic N,N-dialkylated 2-aminotetralins; trans-2-phenylcyclopropylamines; (+)-cis-8-hydroxy-1-methyl-2-(di-n-propylamino)tetralin; (1R,2S)-2-(2-hydroxyphenyl)-N,N-di-n-propylcyclopropylamine [(1R,2S)-4]; and a 3-hydroxy isomer (1R,2S)-5;8-hydroxy-2-(dipropylamino)tetralin; cis or trans-1,2,3,4,4a,5,10,10a-octahydro-9-hydroxy-1-propylbenzo[g]quinolines; CPDD-0068 2,5-dimethyoxy-4-(n)-propyl-thiophenethylamine; CPDD-066 5-methoxy-N-N-diisopropyltryptamine; (2R,3S)-8-hydroxy-3-methyl-2-(dipropylamino)tetralin; Pramipexole; (S)-2-amino-4,5,6,7-tetrahydro-6-(propylamino)benzothiazole salts and isomers thereof. In certain embodiments the agent is administered in an effective amount.

In particular embodiments, patients receive agents such as but not limited to paroxetine, fluoxetine, sertraline, venlafaxine, tricyclics, or duloxetine for a period of six months or more, and are then evaluated for the presence of the anhedonic symptoms defined in the following paragraphs.

Along with decreased dopaminergic availability and/or transmission efficiency resulting from ongoing administration of one or more agents that increase the availability and/or efficiency of serotonin (with or without increases in the availability or efficiency of norepinephrine), may occur certain psychiatric symptoms. The exemplar clinical finding in the anhedonic symptom complex defined herein is a sense that life is experienced as irremediably “dull” and characterized by what one trained in the art will understand as significant diminution in or apparent functional absence of the positive affect involved in normal attention to novel stimuli—the range from mild interest to ordinary excitement over their normal range.

Although one skilled in the art might expect any symptom produced by a drug to abate when such treatment is discontinued, once a patient has as a sequella of serotonergic therapy experienced the symptoms defined herein as treatment emergent or iatrogenic anhedonia, the anhedonic symptoms will, in certain embodiments, continue to require treatment.

The patients may present with continued and apparently adequate control of the painful depressive symptoms for which the medication had originally been prescribed, despite that one skilled in the art may elicit from such patients a significant number of symptoms described herein and defined as anhedonia. Patients undergoing chronic serotonergic therapy can be identified as subject to the herein defined anhedonia despite their freedom from the unpleasant symptoms for which they had originally sought treatment.

In some embodiments, standard clinical evaluation will provide assurance that prior to the development of the psychiatric illness requiring serotonergic and/or adrenergic treatment, nothing about the life or behavior of the patient had suggested the diagnosis of Attention Deficit Disorder or Attention Deficit Hyperactivity Disorder (ADD/ADHD).

In certain embodiments, iatrogenic anhedonia appears de novo, typically after six months or more of serotonergic therapy. Although it may appear somewhat earlier, and certainly may be demonstrated to appear during any later period characterized by the continuous maintenance of such treatment targeting the increased efficiency and availability of serotonin with or without similar increases in the efficiency and availability of norepinephrine, this general timeframe for the appearance of treatment emergent anhedonia provides a unique clinical condition different from any previously reported condition, and is not a late appearing resistance to or failure of serotonergic treatment. As the administration of SSRIs and SNRIs can produce an increase in DAT, any so caused increase in DAT will produce co-incident decreases in dopaminergic transmission efficiency while serotonin transmission efficiency or the combination of serotonin and norepinephrine transmission efficiency is increased, as explained further below.

Anhedonic patients may also be identified from among those persons who spontaneously exhibit such symptoms, as well as those having undergone successful and chronic treatment for major depressive disorder with medications that inhibit neuronal reuptake of both serotonin and one or more other neurotransmitters. The herein defined anhedonia symptoms are likely to appear when any agent or agents have acted to decrease dopaminergic transmission efficiency. Any set of patients in whom a treatment produces a decrease in dopaminergic transmission efficiency will exhibit the herein defined syndrome of iatrogenic anhedonia.

### A. Characteristics of Anhedonia

Anhedonia (iatrogenic or spontaneous) includes a cluster of psychological symptoms characterized by the absence of specific pleasant normal emotions rather than the presence of specific unpleasant normal or pathological emotions. Anhedonic individuals note some or all of the following symptoms: 1) a reduction in or a stable inability to experience the range of feelings from normal interest to pleasurable excitement in reaction to ordinary life experiences; 2) slow but steadily worsening loss of “energy” associated with significant reduction in the search for novel experiences normally expected to produce such pleasant feelings; 3) routine activities are performed with no sense of reward, relief, or joy at their completion; 4) when questioned, admission to a gradually increasing sense of boredom or ennui, despite that they do not seek activities normally associated with relief of such complaints; 5) decrease or apparent loss of interest in personal advancement; 6) a sense of a lack of amusement associated with events and entertainments described by others as vehicles for laughter and fun.

Accordingly, as noted above, anhedonic patients may be identified, as one example, from among those having undergone successful and/or chronic treatment (six months or longer) for a major depressive disorder with any medication that increases serotonin availability and/or efficiency, or that increases the synthesis and release of serotonin, including but not limited to the Selective Serotonin Reuptake Inhibitors (SSRIs) or Serotonin Norepinephrine Reuptake Inhibitors (SNRIs) at any dosage.

In certain instances, for ease of identification purposes, an anhedonic patient is preferably a patient who has undergone treatment for a major depressive disorder as described previously and who exhibits one or more of the anhedonic psychological symptoms set forth above. In this subset, such treated patients may present with continued and apparently adequate control of the specific depressive symptoms for which the medication had originally been prescribed, despite that one skilled in the arts related to the present invention may elicit a significant number of the new dysphoric symptoms described herein and defined as an iatrogenic anhedonia.

In those instances where a patient exhibits iatrogenic anhedonia, it is also recognized that iatrogenic anhedonia may be induced by such medications when chosen for therapeutic reasons by one practiced in the art of psychopharmacology to produce chronic increases in serotonergic transmission efficiency, resulting in decreased dopaminergic transmission efficiency in conditions other than major depressive disorder. Specifically, in certain embodiments the present invention is equally applicable to cases in which such treatment has been prescribed for any other known or yet to be discovered condition, such as, but not limited to obsessive compulsive disorder, obsessive compulsive personality disorder, social phobia, premature ejaculation, and premenstrual dysphoric disorder.

In certain embodiments, iatrogenic anhedonia symptoms may be produced in other conditions that are now or may in the future be treated by chronic blockade of SERT, by SSRI or SNRI therapy. Accordingly, one of skill in the relevant art understands in light of the present disclosure that iatrogenic anhedonia symptoms may be produced by any future therapies that cause upregulation, and in particular chronic upregulation, of serotonergic transmission efficiency or increased synthesis and/or release of serotonin with decreased dopaminergic transmission efficiency.

### B. Differences Between a Major Depressive Disorder and Anhedonia

These psychological complaints of anhedonia differ from those seen in the wide range of depressive illnesses in that they are: 1) unassociated with thoughts about death or morbid preoccupation about the future; 2) are not accompanied by significant distress, inappropriate anger, guilt, shame, or unusual degrees of anxiety; 3) do not interfere with normal sleep; 4) are not associated with significant increase or decrease in body weight; and 5) do not interfere with the ability to perform at one's usual occupation. Individuals suffering from this condition rarely volunteer the symptoms described in the previous paragraph or seek treatment for this cluster of complaints, despite willingness to so disclose and gratitude when questioned appropriately.

Furthermore, significant difference between the clinical presentations of a major depressive disorder and the late-appearing iatrogenic anhedonia described herein may be found in the specific affects expressed by the patients. Even though patients with a major depressive disorder may admit significant decrease in the ability to enjoy positive affect, the complaints that trigger the request for clinical attention involve specific negative (unpleasant) affects, usually the cluster comprised of distress, anger, anxiety, guilt, shame, and self-disgust. In the iatrogenic anhedonia described herein, those negative affects are rarely expressed; rather, after a significant period of freedom from such negative affects and the liberty to enjoy positive affect, the patient now exhibits what seems to be almost purely a reduction in the ability to express positive affect over the range from mild interest to pleasurable excitement, and contentment to joy.

The recognition that by use of these criteria a late-appearing anhedonia can be separated from the initial disease state has led to an understanding that the treatment modality itself is the causative agent of the syndrome herein described, as well as the rationale for the treatment herein defined.

Once identified as having a form of anhedonia, (e.g., iatrogenic anhedonia) the patient can be treated according to the methods of the present invention as detailed hereinafter.

### II. Methods of Treatment of Anhedonia

Once a patient is diagnosed with a form of anhedonia, in certain embodiments the treatment regimen of the present invention includes the administration of one or more agents capable of directly and/or indirectly increasing the availability of dopamine and/or directly or indirectly increasing the efficiency of dopaminergic activity. In one embodiment, the agent or agents used to treat anhedonia are administered while a patient is undergoing treatment for a major depressive disorder.

Specifically, patients diagnosed as exhibiting anhedonia are treated with an effective amount of a pharmaceutically active agent, where the amount of agent and type of agent is sufficient to induce, facilitate, or improve dopaminergic availability and/or neurotransmission efficiency. Compounds that facilitate dopaminergic transmission include dopamine analogues, direct dopamine agonists, indirect dopamine agonists, compounds that induce transcription of genes encoding dopamine receptors, any agent or agents that increase the synthesis and/or release of dopamine, compounds that inhibit transcription of genes encoding DAT, compounds that inhibit dopamine binding by DAT, and/or agents that act through dopamine receptors by either direct and/or indirect action to enhance the efficiency of dopaminergic transmission. For patients diagnosed and treated using the present methods, one practiced in the art can thereby facilitate the return to a state in which positive affect can be triggered by stimuli generally considered to be ordinary and normal by one practiced in the art, and such affect mobilized over its normal range.

Accordingly, the present invention addresses (1) patients exposed to the chronic use of SSRI or SNRI medications who do now and/or will in the future suffer degradation of treatment response as a direct consequence of alterations in dopaminergic transmission efficiency induced by whatever root cause, as well as (2) the significant fraction of patients who spontaneously (e.g., without chronic or significant prior exposure to an SSRI or SNRI) exhibit or develop anhedonic symptoms.

As described above, in certain embodiments, the present invention defines a symptom pattern as treatment emergent anhedonia, or “iatrogenic anhedonia,” resulting from pharmacological treatment of a patient with an agent or agents that increase serotonin transmission efficiency with or without similar increases in the efficiency and availability of norepinephrine.

### A. Therapeutic Agents for Use in Treatment

In certain embodiments of the present invention, diagnosis and treatment of anhedonia, and in particular iatrogenic anhedonia, is the counterintuitive observation that intermittent and/or low doses of dopaminergic medication provide the most effective treatment for this condition.

Therapeutic regimens useful in the treatment of the anhedonia described herein and/or secondary to the prolonged use of agents that increase the availability and/or efficiency of serotonergic transmission, may involve any agent (including but not limited to synthesized compounds or those derived from natural sources) that 1) increases the availability of dopamine or 2) increases dopaminergic efficiency by enhancement of dopamine receptor activation; 3) inhibits the removal of dopamine; 4) optimizes the ability of dopamine to activate dopamine receptors; 5) agents that produce elevations in the synthesis and/or release of dopamine; or 6) any combination of these identified mechanisms that enhances dopaminergic transmission efficiency. Such therapeutic regimen may also include a combination of any or all of these agents. The agents may be administered orally, parenterally, by cutaneous application, and/or by inhalation in whatever formulation produces the normalization or optimization of mood.

In certain embodiments, some agents useful for the treatment of anhedonia include one or more stimulants. It should be noted however, that the prescribing information for stimulants explicitly defines such drugs as contraindicated in the treatment of major depressive disorders. In certain embodiments, agents useful for the treatment of anhedonia include one or more of the following: AC-90179 [2-(4-Methoxyphenyl)-N-(4-methyl-benzyl)-N-(1-methyl-piperidin-4-yl)-acetamide Hydrochloride; (1S,2R)-8-Hydroxy-1-methyl-2-(dipropylamino)tetralin; cis-(1S,2R)-8-hydroxy-1-methyl-2-(di-n-propylamino)tetralin or monophenolic N,N-dialkylated 2-aminotetralins and trans-2-phenylcyclopropylamines; (+)-cis-8-hydroxy-1-methyl-2-(di-n-propylamino)tetralin; (1R,2S)-2-(2-hydroxyphenyl)-N,N-di-n-propylcyclopropylamine [(1R,2S)-4] and its 3-hydroxy isomer (1R,2S)-; 8-hydroxy-2-(dipropylamino)tetralin; cis or trans-1,2,3,4,4a,5,10,10a-octahydro-9-hydroxy-1-propylbenzo[g]quinolines; (2R,3S)-8-hydroxy-3-methyl-2-(dipropylamino)tetralin; Ropinirole; (4-[2-(dipropylamino)ethyl]-1,3-dihydro-2H-indole-2-one; Pramipexole; (S)-2-amino-4,5,6,7-tetrahydro-6-(propylamino)benzothiazole; ritalin; methyl 2-phenyl-2-(2-piperidyl)acetate; Focalin; 2-phenyl-2-(2-piperidyl)acetic acid; methylphenidate; methyl 2-phenyl-2-(2-piperidyl)acetate hydrochloride; d-threo-methylphenidate; amphetamine; tianeptine; [3-chloro-6-methyl-5,5-dioxo-6,11-dihydro-(c,f,)-dibenzo-(1,2,-thiazepene)-11-yl)] amino]-7 heptanoic acid; Tiagabine; (−)-(R)-1-[4,4-Bis(3-methyl-2-thienyl)-3-butenyl]nipecotic; nicotine; L-DOPA clozapine; donepezil; tiagabine; gabapentine; pentoxifylline; modafanil; levocamitine; piracitam; mazindol; topiramate; mianserin; isradapine; olnazapine; tolcapone; ciproxifan; thioperimide; chlobenpropit; Ketamine; R-(+)-3-Allyl-6-bromo-7,8-dihydroxy-1-phenyl-2,3,4,5-tetrahydro-1H-3-benzazepine [(R)-(+)-6-Br-APB, 18; amantadine; aripiprazole; fenoldopam 2,6-dibenzylidenecyclohexanone; 2,6-dibenzylidenecyclohexan-1-one; 2,6-dibenzylidene-cyclohexanone; trans-6,6a,7,8,9,13b-hexahydro-5H-benzo[d]naphth[2,1-b]azepines as rigid analogues of beta-phenyldopamine is 9-Dihydroxy-2,3,7,11b-tetrahydro-1H-naph[1,2,3-de]isoquinoline; dihydrexidine [4;(+/−)-trans-10,11-dihydroxy-5,6,6a,7,8,12b-hexahydrobenzo[a]phenanthridine; trans-10,11-dihydroxy-5,6,6a,7,8,12b-hexahydrobenzo[a]phenan thridine (4a, dihydrexidine; trans-hexahydroindolo[4,3-ab]phenanthridines (“benzergolines”); (+/−)-trans-2-Methyl-10,11-dihydroxy-5,6,6a,7,8,12b-hexahydrobenzo[a]phenanthridine; cis- and trans-9- and 11-hydroxy-5,6,6a,7,8,12b-hexahydrobenzo[a]phenanthridines; (+/−)-3-[4′-(N,N-dimethylamino)cinnamyl]benzazepine; 1-phenyl-, 4-phenyl-, and 1-benzyl-1,2,3,4-tetrahydroisoquinolines; 7,8-dihydroxy-1-phenyl-2,3,4,5-tetrahydro-1H-3-benzazepine(SKF 39383); SKF 82958; ABT 431; (5R)-8-chloro-2,3,4,5-tetrahydro-3-methyl-5-phenyl-1H-3-benzazepin-7-oll 2,3,4,5-tetrahydro-1-phenyl-1H-3-benzazepine-7,8-diol; cis- and trans-2,3,4,8,9,9a-hexahydro-4-phenyl-1H-indeno[1,7-cd]azepine-6,7-diol; ([1R,3S]-1-aminomethyl-5, 6-dihydroxy-3-phenylisochroman);(N-n-propyl-N-phenylethyl-p-3-hydroxyphenylethylamine; 4aR,10bR)-(+)-trans-3,4,4a,10b-tetrahydro-4-n-propyl-2H,5H [1]benzopyrano[4,3-b]-1,4-oxazin-9-ol; 5- and 7-hydroxy-2-(di-n-propylamino)tetralin and cis- and trans-7-hydroxy-4-n-propyl-1,2,3,4,4a,5,6,10b-octahydrobenzo [f]quinoline; cis- and trans-4-n-propyl-1,2,3,4,4a,5,6,10b-octahydrobenzo[f]quinolines; (+/−) 8-hydroxy-2-(di-n-propylamino)tetralin; Pramipexole; (S)-2-amino-4,5,6,7-tetrahydro-6-(propylamino)benzothiazole; (+/−)-2,5-demethoxy-4-iodoamphetamine (DOI); topiramate (topomax); 2,3:4,5-Di-O-isopropylidene-beta-D-fructopyranose sulfamate; PRX-00023; PRX03140; PRX-07037; aripiprazole; resperidone; mirtazapine; flumazenil; prinepirone; CPDD-0063 1-Benzylpiperazine difumarate; CPDD-0068 2,5-dimethyoxy-4-(n)-propyl-thiophenethylamine; CPDD-066 5-methoxy-N-N-diisopropyltryptamine; CPDD-066 5-methoxy-N-N-diisopropyltryptamine; tropane alkaloids which have the 8-azabicyclo[3.2.1]octane nucleus; alfentanil; ropinirole; quetiapin; and isomers, enantomers, salts and derivatives thereof.

In certain embodiments suitable agents can include one or more of methylphenidate, dexmethylphenidate, amphetamine salts, bupropion, nefazodone, and aripiprazole, ropinirole, quetiapene; isomers, enantiomers, salts, and derivatives thereof. In the present invention, these agents may be used alone in the treatment of anhedonia or one of the agents may be combined with one or more other agents.

In one embodiment of the present invention, a treatment regimen includes the continuation and possible downward adjustment of the originally prescribed serotonergic medication as it is supplemented by the dopaminergic pharmacotherapy.

In certain embodiments, the present invention requires that the one or more agents capable of directly and/or indirectly increasing the availability of dopamine and/or directly or indirectly increasing the efficiency of dopaminergic activity be administered according to the examples provided herein.

However, in other embodiments, the present invention includes one or more of such agents administered in reduced amounts and/or with reduced regularity, as explained below. In certain embodiments, the agent can be administered to a patient at a dose that produces a clinical effect (i.e., an effective amount) and thus ameliorates the anhedonic symptoms. In certain embodiments, the dose of the agent capable of directly and/or indirectly increasing the availability of dopamine and/or directly or indirectly increasing the efficiency of dopaminergic activity, and thusly producing a clinical effect, can be administered at significantly reduced amounts, including about 2%, 2.5%, 3%, 5%, 7%, 10%, 12.5%, 15%, 20%, 25%, 35%, 45%, 50%, 60%, 70%, 80%, 90% or 95% of the dose, normal or maximum, that according to the prescribing standards would be administered to a patient in the absence of a major depressive disorder (see, e.g, Physicians Desk Reference, 6th Ed) and having a condition that is treatable by the administration of a dopaminergic agent, alone or in combination with one or more other agents. In certain embodiments, the amount is about 2% to 90% of the maximum or normal dose. In other embodiments, about 2% to 50% may be administered. In yet another embodiment, about 15% to 50% may be administered.

In certain embodiments, the dose administered is about 2.5%, 3%, 5%, 10%, 25% or 50% of the maximum daily dose. In other embodiments, any dose of an agent capable of directly and/or indirectly increasing the availability of dopamine and/or directly or indirectly increasing the efficiency of dopaminergic activity that is administered to a patient for the treatment of anhedonia in a treatment regimen of the invention is about 1, 2, 3, 4, 5, 6, 7 or 8 times the amount of the reduced dose efficacious for the treatment of anhedonia. In certain embodiments, the agent is in an immediate, controlled or sustained release form.

The amount of agent or agents administered to a patient may be modulated, i.e., up-titrated or down-titrated, based on clinical observation consistent with the observations and teachings set forth herein. In certain embodiments, each week the dose of agent or agents is up-titrated or down-titrated by an amount equal to the initial dose (e.g., initial dose at week one: 5 mg., week 2: 10 mg., week 3: 15 mg., week 4: 20 mg.).

In yet another embodiment, the methods of the invention can be used for the discovery of any agent, dosing amount, and dosing schedule of an agent that enhances the ability of dopamine to stimulate activity through either direct and/or indirect mechanisms of action. Specifically, once a patient is diagnosed with a form of anhedonia, iatrogenic or spontaneous, a drug that exhibits effectiveness in treating said anhedonia may be classified as an agent that enhances the ability of dopamine to stimulate activity through either direct and/or indirect mechanisms of action. The prediction of agents enhancing dopaminergic transmission efficiency therefore facilitates a method for the effective identification and development of agents and modalities useful in the amelioration of any such anhedonia, whether caused by the prolonged upregulation of serotonin transmission efficiency or otherwise.

In one embodiment, the present invention includes a dosage form that is a combination of any medication that increases serotonin availability and/or efficiency, or that increases the synthesis and release of serotonin (e.g., one or more of the drugs set forth in Part I, above) and a medication that is useful for the treatment of anhedonia.

In certain embodiments, the present invention includes a kit providing appropriate instructions to a patient. In certain other embodiments, the instructions provide dosing schedules.

### B. Dosing Schedules

In certain embodiments, up- or down-titration may continue until resolution of a patient's anhedonia. Although the agent or agents may be administered daily, in certain embodiments, the agent or agents are administered according to the experience of one practiced in the art.

Development of dosing schedules as well as dosing amounts for providing relief of anhedonia, and in particular iatrogenic anhedonia, can be complex. However such development is routine and well within the skill of one in the art in light this disclosure. In one embodiment, schedules may require frequent modulation based upon clinical assessment by one practiced in the art of psychopharmacology in view of the teachings herein. For example, the dosage of methylphenidate may require frequent modulation to produce the herein defined therapeutic effect (i.e., the return of the normal range of what is consensually defined as pleasant emotion or positive affect). Additional improvement in or normalization of mood may follow specific substitution or addition of adjuvant agents such as but not limited to amphetamine salts, ropinirol, quetipine, aripiprazole, or any agent producing an enhancement of dopaminergic transmission efficiency, or formulations that enable long acting modulation of dopaminergic transmission efficiency.

A treatment regimen of the present invention may include the continuation and possible downward adjustment of the originally prescribed serotonergic medication as it is supplemented by the dopaminergic pharmacotherapy. For example, therapeutic regimens may involve doses of bupropion that can range from about 150-300 mg/day through whatever modes of administration that produce the best clinical effect, or nefazodone in doses ranging between about 25 to 300 mg/day through whatever modes of administration that produce the best clinical effect. Clinical judgment normal to one skilled in the art of psychopharmacology will determine the need for or the benefit to accrue from downward titration of the prior serotonergic therapy.

Likewise, therapeutic regimens may involve amphetamine salts or any derivative thereof prescribed from about 2.5 mg to 10 mg/day in whatever modes of administration that produce the best clinical effect. Similarly, therapeutic regimens may involve either methylphenidate or its derivatives prescribed over an analogous range.

Satisfactory and ongoing improvement is provided by doses as low and sporadic as, in but one example, about 2.5 mg of dexmethylphenidate taken on a prn basis no more than once daily on as few as two to five days per week in whatever modes of administration that produce the best clinical effect.

Advantageous treatment regimens for the iatrogenic anhedonia defined herein include those that result in an increase of dopamine transmission efficiency by any or all of direct and/or indirect action on dopamine sensitive neurons, inhibition of the dopamine transporter, the induction of chronic elevations in dopamine availability, the induction of transient increases in dopamine availability and/or efficiency, and increases in dopamine transmission efficiency by any mechanism.

The pharmaceutical substances of the invention can be administered orally, parenterally, by cutaneous application, and/or by inhalation in whatever formulation produces the mood elevation or normalization.

### III. Relation Between Anhedonia and Serotonin

While the invention is not limited to any particular hypotheses, it is believed that medications which increase serotonin transmission efficiency with or without similar increases in the efficiency and availability of norepinephrine, such as SSRIs or SNRIs, may at some time during such treatment lead to anhedonia by decreasing the efficiency of dopaminergic transmission. In some instances, the long term administration of an antidepressant leads to a permanent alteration of a patient's brain chemistry that cannot be resolved or ameliorated by cessation of the antidepressant therapy. This is supported by the finding disclosed herein that agents capable of increasing dopaminergic efficiency are useful in the treatment of this specific anhedonia secondary to prolonged treatment that produces increases in the efficiency of serotonergic transmission with or without increases in adrenergic transmission and/or efficiency.

As one specific example, methylphenidate, which is known to inhibit DAT, may be used to treat the condition. Other agents effectuating increased dopaminergic transmission efficiency, such as but not limited to dexmethylphenidate, amphetamine salts, bupropion, nefazodone, and aripiprazole may also find use as treatments leading to normalization of the ability to experience pleasure as defined herein.

For such an anhedonic condition as defined herein it is further postulated (without limitation arising from such postulation) that this anhedonia, emerging as a late-appearing result of treatment with SSRIs, SNRIs and other agents that produce chronic increases in serotonin availability and/or transmission efficiency with or without analogous increases in noradrenergic transmission efficiency, may be ameliorated by treatment with agents effectuating increased dopaminergic activity and/or efficiency, and that such diagnosis and treatment may be more specifically defined and facilitated by the use of appropriate genetic and psychological tests.

It would be understood by one of ordinary skill in the art that improvement in diagnostic algorithms can provide important benefits to society. Optimally formulated case/disease management programs can improve detection and decrease duration of disability, thus improving the quality of life for patients and encouraging reduction in fully burdened costs for the treatment of such patients. Such benefits are provided by the diagnosis of and treatment for anhedonia defined herein.

A retrospective review of patient data indicates that anhedonic symptoms can easily be demonstrated in a cohort of patients receiving SSRI or SNRI treatment for major depressive disorder for a period between six months and two years or more. Although in the majority of so afflicted patients the diagnosis will be apparent during this interval, it should be understood that iatrogenic anhedonia may appear earlier or later during any course of treatment with medications that increase serotonin transmission efficiency with or without similar increases in the efficiency and availability of norepinephrine. The rather substantial number of patients suffering from the condition over diverse population samples may be postulated to be due to a genetic predisposition for the condition.

With respect to the table below, it is shown that prolonged treatment of patients with medications producing constant elevations of or increases in the efficiency and/or availability of serotonin, with or without analogous increases in the efficiency and/or availability of norepinephrine, causes such patients to suffer from anhedonia, and that these patients exhibit a marked return to or normalization of interest in ordinary stimuli and pleasure in activities from which it is usually experienced following treatment with agents effectuating enhancement of dopaminergic availability and/or efficiency.

Patients in whom SSRIs produced excellent remission of major depressive disorder for the period shown in the 5^{th} column

| | | | | SSRI | | | | |
|---|---|---|---|---|---|---|---|---|
| Gender, | | | | duration | | All | | Dopamin-ergic |
| Age at | Diagnosis | | Complaint requiring | before new | | subsequent | Current | supplementation |
| entry | (DSM-IV-TR) | SSRI dose, duration | treatment change | symptoms | | Rx | Rx | history |
| Female, | MDD | citalopram | Anomie, fear that | 6 | months | bupropion | nefazodone | mph 18 mg LA |
| 40 | | 10-20 mg; 6 months | previous symptoms | | | nefazodone | gabapentin | daily |
| | | | might recur | | | gabapentin | mph | 2 years |
| | | | | | | mph | | |
| Male, 51 | MDD | fluoxetine | Anomie, boredom | 1 | year | bupropion | nefazodone | mph 18 mg LA |
| | OCP | 20-60 mg; 1 year | | | | venlafaxine | mph | daily 4mths; |
| | | | | | | nefazodone | | 20-30 mg LA |
| | | | | | | mph | | 2 years; now 5 |
| | | | | | | | | mg bid prn |
| Male, 48 | MDD | fluoxetine | Inhibition of | 1 | year | fluoxetine | fluoxetine | mph 5 mg bid prn |
| | OCP | 10-40 mg; 10 years | creativity | (10 years | | bupropion | bupropion | bupropion 150 mg |
| | | | | intermittent)* | | mph | mph | |
| | | | | | | lamotrigine | lamotrigine | |
| Female, | MDD | sertraline | Lost interest in work | 2 | years | Venlafaxine | venlafaxine | bupropion 300 mg |
| 40 | | 50-100 mg; 2 years | and relationships | | | 150 mg 2 | bupropion | |
| | | | | | | years; | | |
| | | | | | | Bupropion 5 | | |
| | | | | | | years | | |
| Male, 53 | MDD | fluoxetine | Anomie, loss of | 2 | years | bupropion | nefazodone | nefazodone |
| | | 20-40 mg 2 years; | interest in previously | | | nefazodone | lamotrigine | mph 5 mg bid |
| | | sertraline | pleasant activities | | | gabapentin | mph | 1 month |
| | | 50-100 mg 2 years; | | | | lamotrigine | | 20 mg LA for |
| | | | | | | mph | | 1 month; |
| | | | | | | | | 5 mg bid prn |
| | | | | | | | | 2 years |
| Male, 35 | MDD | Fluoxetine | Inanition; Loss of | 1 | year | bupropion | nefazodone | nefazodone |
| | | 10 years, 20-40 mg | creativity; fitful | (10 years | | citalopram | gabapentin | mph 5 mg bid; |
| | | | sleep | intermittent)* | | gabapentin | mph | now 20 mg |
| | | | | | | nefazodone | | LA +5 mg prn |
| | | | | | | | | at 3pm for 2 years |
| Female, | MDD | paroxetine | Dullness, loss of | 10 | years | bupropion | bupropion | mph 5 mg bid; |
| 41 | | 20-30 mg/d, | focus on novel stimuli | | | trazodone | trazodone | now 30 mg LA |
| | | trazodone | | | | mph | mph | 1 year |
| | | 50 mg hs, 10 years | | | | | | |
| Male 43 | OCP | sertraline | Shift to venlafaxine | 1½ | years | Venlafaxine | Venlafaxine | Mph 5 mg bid |
| | MDD | 50 mg 8 months, | ppt by return of | | | Mph | Mph | |
| | | triflouoperazine | depressive symptoms. | | | | | |
| | | 1 mg prn. | Addition of mph ppt | | | | | |
| | | Sertraline | by dullness, loss of | | | | | |
| | | 100 mg 10 months; | interest in work and | | | | | |
| | | venlafaxine | relationships, | | | | | |
| | | 37.5 mg 3 years, | reduced creativity | | | | | |
| | | 75 mg 6 years | | | | | | |

*Patient stopped Rx each time anomie experienced, resumed when depression became painful, never discussed with treating psychiatrist.

The preceding table indicates that prolonged exposure to elevated levels of serotonin, and/or changes in the levels of its transporter (SERT), and/or increases in the efficiency of that neurotransmitter (with or without increases in the availability or efficiency of norepinephrine), induces a decrease in the transmission efficiency and/or availability of dopamine. The result of decreased dopaminergic efficiency and/or availability may be dysphoric interference with the ability to experience the normal range of positive affect over the range from mild interest to pleasant excitement, and from ordinary contentment to great joy. Such decreased dopaminergic transmission efficiency may be caused by agents that block the serotonin transporter alone, and/or those that block both the serotonin transporter and the norepinephrine transporter, and/or those that increase the synthesis and release of both norepinephrine and serotonin and any mechanism whereby the efficiency of serotonergic transmission is enhanced.

### EXAMPLE 1

As only one of many therapeutic approaches, patients are to maintain constant their intake of all medications prescribed prior to the start of this trial of dopaminergic therapy. Patients are to maintain constant their intake of all medications prescribed prior to the start of this trial of dopaminergic therapy. In the first week, afflicted patients may be given a single daily dose of methylphenidate 5 mg. In each succeeding week, the dose of methylphenidate may be raised as follows until the achievement of a satisfactory clinical result, failure to achieve a satisfactory result, or a dysphoric reaction requiring cessation of such treatment:

| | |
|---|---|
| Week 2 | 5 mg b.i.d. |
| Week 3 | 10 mg b.i.d. |
| Week 4 | 15 mg b.i.d. |
| Week 5 | 20 mg b.i.d. |

Downtitration of methylphenidate will be suggested by the ordinary clinical judgment normal to those skilled in the art of psychopharmacology. Patients will be instructed to notate the duration of action for each dose of medication, and this data will inform the dosage schedule for the eventual prescription. Other alterations of medications prescribed before institution of this regimen may be suggested as normal for one practiced in the art.

### EXAMPLE 2

As only one of many therapeutic approaches, patients are to maintain constant their intake of all medications prescribed prior to the start of this trial of dopaminergic therapy. In the first week, afflicted patients may be given a daily dose of bupropion 150 mg. If, after this period the patient does not describe adequate relief from the symptoms of iatrogenic anhedonia but has experienced no symptoms suggesting that this medication be stopped, added to this regimen will be a once-daily dose of methylphenidate 5 mg. In each succeeding week, the dose of methylphenidate may be raised as follows until the achievement of a satisfactory clinical result, failure to achieve a satisfactory result, or a dysphoric reaction requiring cessation of such treatment:

| | |
|---|---|
| Week 3 | 5 mg b.i.d. |
| Week 4 | 10 mg b.i.d. |
| Week 5 | 15 mg b.i.d. |
| Week 6 | 20 mg b.i.d. |

Downtitration of methylphenidate will be suggested by the ordinary clinical judgment normal to those skilled in the art of psychopharmacology. Patients will be instructed to notate the duration of action for each dose of medication, and this data will inform the dosage schedule for the eventual prescription. Other alterations of medications prescribed before institution of this regimen may be suggested as normal for one practiced in the art.

The above is a detailed description of particular embodiments of the invention. It is recognized that departures from the disclosed embodiments may be made within the scope of the invention and that obvious modifications will occur to a person skilled in the art. Those of skill in the art should, in light of the present disclosure, appreciate that many changes can be made in the specific embodiments which are disclosed herein and still obtain a like or similar result without departing from the spirit and scope of the invention. All of the embodiments disclosed and claimed herein can be made and executed without undue experimentation in light of the present disclosure.

We claim:

1. A method of treatment comprising the steps of:
a. identifying iatrogenic anhedonia in a patient being treated with a first pharmaceutical agent that increases serotonin; and
b. increasing dopaminergic activity in the patient by administrating a second pharmaceutical agent in an amount effective to ameliorate the iatrogenic anhedonia.

2. A method for the treatment of iatrogenic anhedonia in a patient currently being treated for depression by administration of a first pharmaceutical agent known to increase the availability or efficiency of serotonin, the method comprising the steps of:
a. continuing the treatment of the patient with the first pharmaceutical agent known to increase the availability or efficiency of serotonin; and
b. increasing dopaminergic activity in the patient by administering a second pharmaceutical agent in an amount effective to ameliorate the iatrogenic anhedonia.

3. The method of claim 2, wherein the pharmaceutical agent that increases the availability or efficiency of serotonin comprises one or more of fluoxetine, sertraline, paroxetine, duloxetine and venlafaxine

4. The method of claim 2, wherein the patient has been diagnosed with the late appearing Anhedonia.

5. The method of claim 2, wherein the second pharmaceutical agent comprises one or more of an activator of dopamine receptors, a direct or indirect dopamine agonist, a dopamine transport inhibitor, and a compound that increases the synthesis and/or release of dopamine.

6. The method of claim 2, wherein the second pharmaceutical agent comprises one or more of bupropion, methylphenidate, amphetamine salts, nefazodone, and derivatives thereof.

7. A method for the treatment of iatrogenic anhedonia comprising the steps of:
a. identifying a patient having iatrogenic anhedonia;
b. treating the patient with a pharmaceutical agent that enhances dopaminergic availability or efficiency;
c. modulating doses of the pharmaceutical agent that enhances dopaminergic availability or efficiency based upon clinical presentation and therapeutic responses in the patient.

8. The method of claim 7, wherein the pharmaceutical agent causes downregulation of the dopamine transporter.

9. The method of claim 7, wherein the pharmaceutical agent directly or indirectly activates the dopamine receptors.

10. The method of claim 7, wherein the pharmaceutical agent inhibits dopamine transporter activity.

11. The method of claim 7, wherein the pharmaceutical agent comprises one or more of bupropion, methylphenidate, amphetamine, aripiprazole and nefazodone.

12. The method of claim 7 further comprising a flexible dosing schedule of the pharmaceutical agent that involves titration based upon (1) the patient's relief from iatrogenic anhedonia or exacerbation of iatrogenic anhedonia, or (2) the assessment of those skilled in the art of psychopharmacologic therapy.

13. A method of treatment comprising the steps of:
a. identification of iatrogenic anhedonia in a patient who had been treated with a first pharmaceutical agent that increases serotonin efficiency; and
b. increasing dopaminergic activity in the patient by administering a second pharmaceutical agent in an amount effective to ameliorate the iatrogenic anhedonia.

14. The method of claim 1, wherein the patient had been treated with a first pharmaceutical agent for a period of about 6 months.

15. The method of clam 13, wherein the patient had been treated with the first pharmaceutical agent for a period of about 6 months.

16. The method of claim 7, wherein the step of modulating the dose comprises up-titrating or down-titrating the dose.

17. The method of claim 1, wherein the second pharmaceutical agent comprises an agent which increases the availability of dopamine,

18. The method of claim 1, wherein the second pharmaceutical agent comprises an agent which increases dopaminergic efficiency by enhancement of dopamine receptor activation.

19. The method of claim 1, wherein the second pharmaceutical agent comprises an agent which or inhibits the removal of dopamine.

20. The method of claim 1, wherein the second pharmaceutical agent comprises an agent which optimizes the ability of dopamine to activate dopamine receptors.

21. The method of claim 1, wherein the second pharmaceutical agent comprises an agent which produces elevations in the synthesis and/or release of dopamine.
